(19) United States
(12) Patent Application Publication
(10) Pub. No.: US 2015/0064714 A1
(43) Pub. Date: Mar. 5, 2015

(54) Polypeptides containing a modified fragment of the peptide IF1

(71) Applicant: LIFESEARCH, Ramonville Saint Agne (FR)

(72) Inventor: Tiona ANDRIANAIVOMANANJAONA, VILLEMONBLE (FR)

(73) Assignee: LIFESEARCH, Ramonville Saint Agne (FR)

(21) Appl. No.: 14/095,084

(22) Filed: Dec. 3, 2013

(30) Foreign Application Priority Data
Aug. 28, 2013 (FR) 13 58243

(51) Int. Cl. G01N 33/58; G01N 33/573; C07K 14/47

(52) U.S. Cl. CPC G01N 33/582; C07K 14/4703; G01N 33/573; C07K 2319/60; G01N 2333/914; G01N 2500/02
USPC 435/7.4; 530/324; 530/350; 435/18

(57) ABSTRACT

Labelled polypeptide constituted by:
(i) a peptide fragment of 38 to 50 amino acids including a peptide of sequence SEQ ID NO: 1, and
(ii) a label selected from the group constituted by radioactive groups, non-protein fluorophors, protein fluorophors, magnetic particles or a paramagnetic spin label, in order that when the label is a fluorophor protein, the labelled polypeptide further contains a peptide spacer of 3 to 15 amino acids, located between the peptide fragment and the label and use thereof as a diagnostic tool.

The present invention relates to polypeptides containing a modified fragment of IF1.

IF1 is a protein regulating the activity of the mitochondrial ATP synthases.

This enzyme is present in all energy transducer membranes (bacterial, thylakoid and mitochondrial) and produces the major part of the ATP. The proton motive force generated within the respiratory complexes of the mitochondrial internal membrane by oxidation of molecules originating from the metabolism results in the rotation of a central axis (subunit γ) which successively deforms the three catalytic sites (interfaces α/β) of the ATP synthase and thus enables the condensation of ADP and inorganic phosphate to give ATP.

It was recently discovered that ATP synthase could be located in different cell membranes and thus have different roles other than that of cellular fuel producer. Apart from the mitochondrial internal membrane, it can be located in the plasma membrane and have a role in the metabolism of cholesterol but also in immune recognition and in vacuole membranes and have an action on the cell pH.

In certain physiological situations such as anoxia or hypoxia, the proton motive force of ATP synthase can fail and the rotation of the motor reverses as a result; in that case, the ATP synthase functions as an ATPase, functioning in the direction of the hydrolysis of the ATP.

Under these circumstances, the protein IF1, via its binding to the F₁ catalytic domain of ATP synthase, makes it possible to inhibit the hydrolysis of the ATP. The action of this peptide is unidirectional and reversible, as the inhibiting peptide is ejected from its binding site when the energy conditions are reestablished.

The protein IF1 is a soluble peptide, produced naturally in animals, fungi and plants. The bovine (Bos taurus) protein IF1 is constituted by 84 residues; its homologue in yeast (Saccharomyces cerevisiae) has 63 amino acids. Study of the crystalline structure of the peptide IF1 in bovines shows that it is mainly present as an α-helix from amino acid 19 to amino acid 83 (Cabezón et al., EMBO J. 2001 December 17; 20(24): 6990-6996).

The study by Sanchez-Arago and al. (Oncogenesis (2013) 2, e46) indicates that the protein IF1 is overexpressed in certain tumour cells such as the cells of colon, lung, breast and ovarian cancer.

Owing to the deeper understanding of the involvement of ATP synthase, and of IF1 in different pathologies, in particular in different cancers, ATP synthase or the protein IF1 could be used as a biological label in the diagnosis or the monitoring of certain cancers or of other pathologies linked with abnormal activity of ATP synthase or IF1.

However, there is at present no peptide probe enabling the detection of the level of expression of ATP synthase or of the protein IF1 with good sensitivity and high stability.

Bason and al. (J. Mol. Biol. (2011), 406, 443-453) described a series of modified bovine IF1 containing point mutations, the majority of which result in a decrease in the affinity for F₁-ATPase compared to the natural bovine IF1; only the mutation Y33W displays better affinity than the natural protein.

US 2004/0072739 describes a fusion protein comprising (i) an optional epitope label, such as a polyhistidine label, (ii) a sequence enabling the transport of the said fusion protein to the target cells, (iii) optionally, a sequence enabling the transport of the said fusion protein to the target organelles and (iv) a fragment of the protein IF1 of less than 34 amino acids. Only the fragment constituted by amino acid 14 to amino acid 46 of IF1 gives better ATPase inhibiting activity.

It is therefore necessary to provide novel peptide probes displaying high affinity for ATP synthase and capable of being detected by different imaging techniques.

A subject of the present invention is to remedy this deficiency.

A subject of present invention is to propose a labelled polypeptide constituted by a peptide fragment comprising a peptide of sequence SEQ ID NO: 1 and a label.

The said labelled polypeptide of the invention is constituted by:

(i) a peptide fragment of 38-50 amino acids comprising a peptide of sequence SEQ ID NO: 1, and

(ii) a label selected from the group constituted by radioactive groups, non-protein fluorophors, protein fluorophors, magnetic particles or paramagnetic spin labels, provided that when the said label is a protein fluorophor, the said labelled polypeptide further comprises a peptide spacer of 3 to 15 amino acids, located between the said peptide fragment and the said label.

By “label” is meant a chemical or protein compound capable of being detected using an imaging technique, such as for example fluorescence microscopy, single photon emission tomography (SPECT), positron emission tomography (PET) or magnetic resonance imaging (MRI).

By “paramagnetic spin label” is meant a molecule with an odd number of electrons utilised to “label” another, non-paramagnetic molecule for analysis by electron paramagnetic resonance (EPR).

By “labelled polypeptide” is meant a polypeptide bearing a label and which can be distinguished from a polypeptide with no label having the same amino acid sequence using an imaging technique.

The peptide of sequence SEQ ID NO: 1 is a peptide of 38 to 46 amino acids corresponding to the following sequence: Nter-X₁X₂X₃X₄X₅X₆X₇X₈FX₉KREX₁₀AX₁₁EX₁₂X₁₃X₁₄X₁₅X₁₆X₁₇X₁₈X₁₉X₂₀EX₂₁LX₂₂X₂₃L X₂₄X₂₅X₂₆X₂₇X₂₈X₂₉X₃₀X₃₁X₃₂X₃₃X₃₄X₃₅X₃₆X₃₇-Cter.

The amino acids in bold and underlined in the sequence SEQ ID NO: 1 are strictly conserved in all the peptides of sequence SEQ ID NO: 1.

The amino acid X₁ of the sequence SEQ ID NO: 1 is selected from Ala, Ser, Thr, Val and non-natural derivatives thereof. X₁ may be absent from this sequence.

The amino acid X₂ of the sequence SEQ ID NO: 1 is selected from Val, Ile, Ala, Leu and non-natural derivatives thereof. X₂ may be absent from this sequence.

The amino acid X₃ of the sequence SEQ ID NO: 1 is selected from Arg, Lys and non-natural derivatives thereof. X₃ may be absent from this sequence.

The amino acid X₄ of the sequence SEQ ID NO: 1 is selected from Asp, Glu, Ser, Thr and non-natural derivatives thereof. X₄ may be absent from this sequence.

The amino acid X₅ of the sequence SEQ ID NO: 1 is selected from Ala, Ser, Thr, Val and non-natural derivatives thereof.

The amino acid X₆ of the sequence SEQ ID NO: 1 is selected from Gly, Glu, Thr, Asp, Ser and non-natural derivatives thereof.

The amino acid X₇ of the sequence SEQ ID NO: 1 is selected from Gly, Asp, Glu, Ser, Thr and non-natural derivatives thereof.

The amino acid X₈ of the sequence SEQ ID NO: 1 is selected from Ala, Ser, Thr, Val and non-natural derivatives thereof.

The amino acid X₉ of the sequence SEQ ID NO: 1 is selected from Gly, Val, Thr, Glu, Ser, Asp, Ala, Leu, Ile, Asn, Gln and non-natural derivatives thereof.

The amino acid X₁₀ of the sequence SEQ ID NO: 1 is selected from Gln, Lys, Arg, Asn and non-natural derivatives thereof and the non-natural derivatives of His.

The amino acid X₁₁ of the sequence SEQ ID NO: 1 is selected from Glu, Thr, Gln, Asn, Asp, Ser and non-natural derivatives thereof.

The amino acid X₁₂ of the sequence SEQ ID NO: 1 is selected from Glu, Asp, Ser, Thr and non-natural derivatives thereof.

The amino acid X₁₃ of the sequence SEQ ID NO: 1 is selected from the twenty natural amino acids and non-natural derivatives thereof.

The amino acid X₁₄ of the sequence SEQ ID NO: 1 is selected from Tyr, Phe, Trp and non-natural derivatives thereof.

The amino acid X₁₅ of the sequence SEQ ID NO: 1 is selected from Phe, Val, Ile, Ala, Leu and non-natural derivatives thereof.

The amino acid X₁₆ of the sequence SEQ ID NO: 1 is selected from Lys, Arg, Asn, Gln, non-natural derivatives thereof and the non-natural derivatives of His.

The amino acid X₁₇ of the sequence SEQ ID NO: 1 is selected from Ala, Glu, Gln, Asn, Asp, Val, Leu, Ile, Pro and non-natural derivatives thereof.

The amino acid X₁₈ of the sequence SEQ ID NO: 1 is selected from Asn, Gln, Lys, His, Arg and non-natural derivatives thereof.

The amino acid X₁₉ of the sequence SEQ ID NO: 1 is selected from Ala, Ser, Thr, Glu, Asp and non-natural derivatives thereof.

The amino acid X₂₀ of the sequence SEQ ID NO: 1 is selected from Lys, Arg, Asn, Gln and non-natural derivatives thereof and the non-natural derivatives of His.

The amino acid X₂₁ of the sequence SEQ ID NO: 1 is selected from Gln, Lys, Asn, Arg and non-natural derivatives thereof and the non-natural derivatives of His.

The amino acid X₂₂ of the sequence SEQ ID NO: 1 is selected from Ala, Arg, Asp, Glu, Lys, Leu, Ile, Asn, Gln and non-natural derivatives thereof.

The amino acid X₂₃ of the sequence SEQ ID NO: 1 is selected from Ala, His, Leu, Ile, Pro, Lys, Arg, Trp and non-natural derivatives thereof.

The amino acid X₂₄ of the sequence SEQ ID NO: 1 is selected from Lys, Arg, Asn, Gln and non-natural derivatives thereof.

The amino acid X₂₅ of the sequence SEQ ID NO: 1 is selected from Lys, Glu, Arg, Asp, non-natural derivatives thereof and the non-natural derivatives of His.

The amino acid X₂₆ of the sequence SEQ ID NO: 1 is selected from His, Gln, Ser, Thr, Asn, Trp and non-natural derivatives thereof.

The amino acid X₂₇ of the sequence SEQ ID NO: 1 is selected from His, Lys, Arg, Trp and non-natural derivatives thereof. X₂₇ may be absent from this sequence.

The amino acid X₂₈ of the sequence SEQ ID NO: 1 is selected from Glu, Asp and non-natural derivatives thereof. X₂₈ may be absent from this sequence.

The amino acid X₂₉ of the sequence SEQ ID NO: 1 is selected from Asn, Glu, Asp, Gln, Ser, Thr and non-natural derivatives thereof. X₂₉ may be absent from this sequence.

The amino acid X₃₀ of the sequence SEQ ID NO: 1 is selected from Glu, Asp and non-natural derivatives thereof. X₃₀ may be absent from this sequence.

The amino acid X₃₁ of the sequence SEQ ID NO: 1 is selected from Ile, Leu, Val, Ala and non-natural derivatives thereof.

The amino acid X₃₂ of the sequence SEQ ID NO: 1 is selected from the twenty natural amino acids in humans and non-natural derivatives thereof.

The amino acid X₃₃ of the sequence SEQ ID NO: 1 is selected from His, Lys, Arg, Ala and non-natural derivatives thereof.

The amino acid X₃₄ of the sequence SEQ ID NO: 1 is selected from His, Gln, Leu, Glu, Trp, Ile and non-natural derivatives thereof.

The amino acid X₃₅ of the sequence SEQ ID NO: 1 is selected from Ala, Lys, Ser, Arg, Ile, Asp and non-natural derivatives thereof.

The amino acid X₃₆ of the sequence SEQ ID NO: 1 is selected from Lys, Gln, Asn, Ala, Arg, non-natural derivatives thereof and the non-natural derivatives of His.

The amino acid X₃₇ of the sequence SEQ ID NO: 1 is selected from Glu, Lys, Asp, Ser, Arg, Thr, Asn, Gln, non-natural derivatives thereof and the non-natural derivatives of His.

The twenty natural amino acids are Alanine (Ala), Valine (Val), Leucine (Leu), Isoleucine (Ile), Proline (Pro), Serine (Ser), Threonine (Thr), Asparagine (Asn), Glutamine (Gin), Glutamic acid (Glu), Aspartic acid (Asp), Lysine (Lys), Arginine (Arg), Histidine (His), Methionine (Met), Cysteine (Cys), Tryptophan (Trp), Phenylalanine (Phe), Tyrosine (Tyr) and Glycine (Gly).

The non-natural derivatives of His can be any derivative of His known to a person skilled in the art, more particularly selected from: 3-(1-pyrazolyl)-alanine (PYZ1), 3-(2-tetrazolyl)-alanine(TEZA) and 3-(1,2,4-triazol-1-yl)-alanine (TRZ4).

The derivatives of Phe and Tyr can be any derivative of Phe or Tyr known to a person skilled in the art, more particularly selected from: 4-methoxy-phenylalanine (0A1), 4-hydroxymethyl-phenylalanine (4HMP), 4-methyl-phenylalanine (4PH), 3-methyl-phenylalanine (APD), phenylserine (BB8), 3,4-dihydroxy-phenylalanine (DAH), 3-ethyl-phenylalanine (DMP3), momo-phenylalanine (HPE), 3,4-dimethyl-phenylalanine (MP34), 2-methyl-phenylalanine (MPH2), (betaR)-beta-hydroxy-1-tyrosine (OMX) and o-tyrosine (OTYR).

The derivatives of Trp can be any derivative of Trp, in particular selected from: 7-hydroxy-l-tryptophan (OAF), 4-hydroxy-tryptophan (4HT), 5-hydroxy-tryptophan (HRP), beta-hydroxy-tryptophan (HTR), 5-methyl-tryptophan (MTR5), 2-hydroxy-tryptophan (TRO) and 6-hydroxy-tryptophan (TRX).

The derivatives of Met can be any derivative of Met, in particular selected from: ethionine (ESC) and hydroxy-l-methionine (MEO).

The derivatives of Ala, Val, Leu or Ile can be any derivative of Ala, Val, Leu or Ile known to a person skilled in the art, in particular selected from: 2-amino-butyric acid (ABA), 2-aminoheptanoc acid (AHP), diethylalanine (DILE), homoleucine (HLEU), allo-isoleucine (IIL), norleucine (NLE) and norvaline (NVA).

The derivatives of Asn or Gln can be any derivative of Asn or Gln known to a person skilled in the art, in particular selected from beta-hydroxyasparagine (AHB), (2s,4s)-2,5-diamino-4-hydroxy-5-oxopentanoic acid (GHG), glutamine hydroxamate (HGA), 3-methyl-l-glutamine (LMQ), n-methyl-asparagine (MEN), and n5-methyl-glutamine (MEQ).

The derivatives of Lys can be any derivative of Lys known to a person skilled in the art, in particular selected from diaminobutyric acid (DAB), (2s)-2,8-diaminooctanoic acid (HHK) and ornithine (ORN).

The derivatives of Glu and Asp can be any derivative of Glu or Asp known to a person skilled in the art, in particular selected from 3-methyl-aspartic acid (2AS), 4-hydroxy-glutamic acid (3GL), beta-hydroxyaspartic acid (BHD), 3,3-dimethylaspartic acid (DMK), 5-o-methyl-glutamic acid (GME), (3r)-3-methyl-l-glutamic acid (LME), (3s)-3-methyl-l-glutamic acid (MEG), 2s,4r-4-methyl-glutamate (SYM) and 2-aminoadipic acid (UN1).

The derivatives of Cys can be any derivative of Cys, in particular selected from: selenocysteine (SEC) and homocysteine (HCS).

The derivatives of arginine can be any derivative of Arg, in particular selected from 5-methyl-arginine (AGM), c-gamma-hydroxy arginine (ARO), citrulline (CIR), 2-amino-3-guanidinopropionic acid (GDPR), canavanine (GGB) and homoarginine (HRG).

The derivatives of Ser and Thr can be any derivative of Ser and Thr, in particular selected from: 2-amino-5-hydroxypentanoic acid (AA4), allo-threonine (ALO), 3,3-dihydroxy-alanine (DDZ), 4-hydroxy-L-isoleucine(HIL4), (2s,3r)-2-amino-3-hydroxy-4-methylpentanoc acid (HL2), beta-hydroxyleucine (HLU), homoserine (HSER), 3-hydroxy-l-valine (HVA), 4,5-dihydroxy-isoleucine (ILX), 6-hydroxy-l-norleucine (LDO), 4-hydroxy-l-threonine (TH6) and hydroxynorvaline (VAH).

According to the invention, when the label contained in a labelled polypeptide of the invention is non-protein, it can be bound directly to the peptide fragment by covalent bonding.

In a particular embodiment, the radioactive group contained in a labelled polypeptide according to the present invention can be a radioactive isotope directly incorporated into a peptide by direct electrophilic substitution, or a chemical reagent bearing a radioactive isotope, such as the iodinated Bolton-Hunter reagent, N-succinimidyl[2,3-3H]propionate, or N-succinimidyl 4-[¹⁸F].

The radioactive isotopes capable of being used in the labelling of a peptide fragment are known to a person skilled in the art and for example: ³²P, ¹⁵O, ¹²⁵I, ³H, ¹⁴C, ³⁵S, ¹⁸F, ^{99m}Tc, ¹¹¹In, ⁶⁸Ga, ⁹⁰Y, ¹⁷⁷Lu and ¹³¹I can be mentioned.

The labelling method and the suitable radioactive group can be selected on the basis of the physical and biological properties of a peptide fragment to be labelled and/or the utility of the said labelled polypeptide according to the invention and their selection is within the competence of a person skilled in the art. For example, when a peptide has at least one tyrosine or histidine residue, the said peptide can be directly labelled with Na¹²⁵I, in the presence of an oxidising agent. On the other hand, when either the said peptide does not have a tyrosine or histidine, or the modification of these residues risks affecting the biological activity of the said peptide, or the said peptide is sensitive to oxidation, then the iodinated Bolton-Hunter reagent can be used to indirectly incorporate a ¹²⁵I on the side-chain of a lysine in the said peptide or on the alpha-amino function of the said peptide (The Protein Protocols Handbook, (2002), pp 969-970).

The labelling of a peptide with tritium (³H) also makes it possible not to affect the physical properties and the biological activities of the said peptide, and can be carried out by direct substitution or according to an indirect labelling method via a compound bearing tritium, such as N-succinimidyl[2,3-3H]propionate (3H-NSP) (Müller and al., J. Cell Sci. (1980), 43: 319-28).

It is known that a short half-life isotope, such as ¹⁸F or ⁶⁸Ga, is suitable for the labelling of a peptide, for use in positron emission tomography (PET) (Roosenburg and al., Amino Acids. (2011) November; 41(5): 1049-1058).

A long half-life isotope, such as ⁹⁰Y, ¹⁷⁷Lu or ¹³¹I, is suitable for labelling a peptide used in peptide receptor radiotherapy (Roosenburg and al., Amino Acids. (2011) November; 41(5): 1049-1058).

According to the invention, when the fluorophor contained in a labelled polypeptide according to the invention is non-protein, then it can be selected from the derivatives of xanthene, the cyanines, the derivatives of naphthalene, the derivatives of coumarin, the oxazines, the derivatives of pyrene, and the derivatives of acridine.

As derivatives of xanthene, fluorescein, rhodamine B, rhodamine 6G and tetramethylrhodamine can for example be mentioned.

In the cyanines family, Cy3, Cy5 and indocyanine green can for example be mentioned.

As fluorescent derivatives of coumarin, aminomethyl coumarin acetic acid and 6,8-difluoro-7-hydroxycoumarine can for example be mentioned.

As fluorescent derivatives of naphthalene, 2-dimethylamino-5-sulphonylnaphthalene hydrochloride, 2-dimethylamino-6-sulphonylnaphthalene hydrochloride and 5-dimethylamino-1-sulphonylnaphthalene hydrochloride can for example be mentioned.

As fluorescent derivatives of acridine, proflavine can for example be mentioned.

According to the invention, the non-protein fluorophor can be incorporated into a peptide fragment using different functional groups, such as the isothiocyanate group (-N=C=S), the N-hydroxy-succinimidyl ester group (NHS), the maleimide group, a haloacetyl group or the pyridyldithiol group. The isothiocyanate group or the NHS group can react with a primary amine of a peptide, which can be for example the epsilon amino group of a lysine, or the amino group located at the N-terminal end of the said peptide. The maleimide group, the haloacetyl group or the pyridyldithiol group reacts with the thiol group of a cysteine. When the peptide to be labelled does not contain cysteine, a cysteine can be added at the N-terminal or C-terminal end of the said peptide by genetic engineering, such as the directed mutagenesis PCR technique or cloning into an expression vector previously containing the codon for cysteine before or after the insertion site. All these techniques form part of the general knowledge of a person skilled in the art.

Magnetic particles or a paramagnetic spin label can also be bound to a peptide fragment of the invention via a cysteine.

According to the invention, MTSL (S-(2,2,5,5-tetramethyl-2,5-dihydro-1H-pyrrol-3-yl)methyl methanesulphonothioate) can be mentioned as a paramagnetic spin label contained in a labelled polypeptide of the invention.

When the peptide fragment contains no cysteine, the latter can be introduced into the said peptide fragment at strategic positions by the PCR technique. Advantageously, a cysteine is inserted into the N-terminal region, the C-terminal region or the median region of the said peptide fragment.

According to the invention, a label protein contained in a labelled polypeptide of the invention is a fluorophor protein of about 20 kDa selected from GFP (Green Fluorescent Protein), eGFP (enhanced Green Fluorescent Protein), BFP (Blue Fluorescent Protein), CFP (Cyan Fluorescent protein), YFP (Yellow Fluorescent protein), dsRed, mRFP1, HcRed1, PA-GFP (photoactivatable GFP) or the Kaede protein.

The proteins eGFP, BFP, CFP, YFP or PA-GFP are mutant proteins of the GFP wild protein.

The proteins dsRed and HcRed1 are fluorescent proteins derived from coral and emit a red fluorescence. The protein mRFP1 is a mutant of dsRed.

The Kaede protein is a photoactivatable fluorescent protein derived from coral.

The amino acid sequence and the excitation and emission wavelength of these fluorescent proteins are known in the prior art.

According to the invention, a labelled polypeptide constituted by a peptide fragment and a label protein can be produced by any genetic methods known to a person skilled in the art, in particular by the cloning of a nucleic acid encoding a peptide fragment of the invention into an expression vector into which the coding nucleic acid, a fluorophor protein and a spacer is previously inserted, or by the cloning of a nucleic acid constructed by PCR encoding a peptide fragment, a peptide spacer and a fluorophor protein.

According to the invention, the label protein is bound to a peptide fragment of the invention using a peptide spacer of 3 to 15 amino acids.

In a particular embodiment, when a labelled polypeptide according to the invention contains a spacer and when the said spacer is located at the C-terminal end of the said peptide fragment, the said peptide spacer is an oligopeptide of 3 to 5 amino acids.

Among the suitable spacers, the spacer “GlyGlySerGly” (SEQ ID NO: 21) can for example be mentioned.

In another particular embodiment, when a labelled polypeptide according to the invention contains a spacer and when the said spacer is located at the N-terminal end of the said peptide fragment, the said peptide spacer is an oligopeptide of 9 to 15 amino acids.

For example, such a spacer can be “GlyGlyGlyGlySer GlyGlyGlyGlySer GlyGlyGlyGlySer” (SEQ ID NO: 22).

The peptides of sequence SEQ ID NO: 2, 3 or 4, which are respectively a fragment of yeast, bovine or human IF1, are peptides corresponding to the consensus sequence SEQ ID NO: 1.

In a particular embodiment of the invention, the peptide fragment in a labelled polypeptide of the invention comprises or is constituted by:

(i) a peptide mutated relative to a fragment of yeast IF1 of sequence SEQ ID NO: 2, the said mutated peptide comprising at least one mutation at position 15, 27, 34 or 37 as defined relative to the 1s^{t} amino acid of the sequence SEQ ID NO: 2, or

(ii) a peptide mutated relative to a fragment of bovine IF1 of sequence SEQ ID NO: 3, the said mutated peptide comprising at least one mutation at position 19, 31, 42 or 45 as defined relative to the 1^{st} amino acid of the sequence SEQ ID NO: 3, or

(iii) a peptide mutated relative to a fragment of human IF1 of sequence

SEQ ID NO: 3, the said mutated peptide comprising at least one mutation at position 19, 31, 42 or 45 as defined relative to the 1^{st} amino acid of the sequence SEQ ID NO: 4.

The peptides of sequence SEQ ID NO: 5, 6, 7, 8, 9 and 10, which contain a mutation relative to the peptide of sequence SEQ ID NO: 2, the peptides of sequence SEQ ID NO: 11, 12, 13, 14 and 15, which contain a mutation relative to the peptide of sequence SEQ ID NO: 3, and the peptides of sequence SEQ ID NO: 16, 17, 18, 19 and 20, which contain a mutation relative to the peptide of sequence SEQ ID NO: 4 can be non-exhaustively mentioned.

The origin and the position of the mutation of these peptides are stated in the following table.

TABLE 1

| Yeast | | Bovine | | Human | |
|---|---|---|---|---|---|
| SEQ ID NO: 5 | K34A | SEQ ID NO: 11 | H42K | SEQ ID | H42K |
| | | | | NO: 16 | |
| SEQ ID NO: 6 | K37A | SEQ ID NO: 12 | K45A | SEQ ID | K45A |
| | | | | NO: 17 | |
| SEQ ID NO: 7 | H27R | SEQ ID NO: 13 | A31R | SEQ ID | A31R |
| | | | | NO: 18 | |
| SEQ ID NO: 8 | H27A | SEQ ID NO: 14 | A31H | SEQ ID | A31H |
| | | | | NO: 19 | |
| SEQ ID NO: 9 | F15W | SEQ ID NO: 15 | R19F | SEQ ID | R19F |
| | | | | NO: 20 | |
| SEQ ID NO: | F15A | | | | |
| 10 | | | | | |

In an advantageous embodiment of the invention, a labelled polypeptide of the invention is constituted by a peptide fragment of 38 to 50 amino acids comprising or constituted by a peptide of sequence SEQ ID NO: 5, 6, 7, 8, 9, 10, 11, 12, 13, 14, 15, 16, 17, 18, 19 or 20, or a sequence having at least 80% similarity with an aforesaid sequence, advantageously 90%, yet more advantageously 95%.

By “similarity” between two sequences is meant a percentage of identities and/or of conservative substitutions between these two sequences.

The said percentage can be calculated according to various sequence alignment algorithms known in the prior art. For practical reasons, the similarity between two sequences can be calculated using a sequence alignment program developed on the basis of one or more of these algorithms, for example the FASTA program or the NCBI-BLAST program, Jalview.

When such a program is used, the search parameters are the parameters defined by default by the program developer.

By way of example, when the FASTA program is used, the search parameters are those defined by default on the site http://www.ebi.ac.uk/Tools/sss/fasta/.

Essentially, the parameter “ktup”, which controls the sensitivity and speed of the program, is 2. The matrix used is the matrix BLOSUM50. The default score attributed to the first residue in a gap is −10. The default score attributed to each additional residue in a gap is −2.

By way of example, when the Jalview program is used, the search parameters are those defined by default in the option “autocalculate consensus” for the Jalview program.

In an advantageous embodiment of the invention, a labelled polypeptide according to the invention is constituted, from the N-terminal end to the C-terminal end, by:
(i) a peptide fragment of 38 to 50 amino acids comprising a peptide of sequence SEQ ID NO: 1,
(ii) a peptide spacer of 3 to 5 amino acids, and
(iii) a proteinic fluorophor.

In a more advantageous embodiment of the invention, a labelled polypeptide of the invention is constituted, from the N-terminal end to the C-terminal end, by:
(i) a peptide fragment of sequence SEQ ID NO: 7, 9 or 10,
(ii) a peptide spacer of sequence SEQ ID NO: 21, and
(iii) the protein GFP or dsRed.

In a particularly advantageous embodiment of the invention, a labelled polypeptide of the invention is represented by the sequence SEQ ID NO: 23.

In another advantageous embodiment of the invention, a labelled polypeptide of the invention is constituted, from the N-terminal end to the C-terminal end, by:
(i) a proteinic fluorophor,
(ii) a peptide spacer of 9 to 15 amino acids,
(iii) a peptide fragment of 38 to 50 amino acids comprising a peptide of sequence SEQ ID NO: 1.

In a more advantageous embodiment of the invention, a labelled polypeptide of the invention is constituted, from the N-terminal end to the C-terminal end, by:
(i) the protein GFP or dsRed,
(ii) a peptide spacer of sequence SEQ ID NO: 7, 9 or 10, and
(iii) a peptide fragment of sequence SEQ ID NO: 22.

In a particularly advantageous embodiment, a labelled polypeptide of the invention is represented by the sequence SEQ ID NO: 24.

Another particular embodiment of the invention relates to a labelled polypeptide constituted by either a peptide fragment of sequence SEQ ID NO: 7 radioactively labelled with a radioisotope selected from tritium, ³²P, ¹⁵O and ³⁵S, or: (i) a peptide fragment of sequence SEQ ID NO: 7 and (ii) a chemical reagent bearing a radioisotope selected from tritium, ³²P, ¹⁵O and ³⁵S.

Another particular embodiment of the invention relates to a labelled polypeptide constituted by: (i) a peptide fragment of sequence SEQ ID NO: 7 and (ii) the fluorophor Dylight® 680.

Another particular embodiment of the invention relates to a labelled polypeptide constituted by: (i) a peptide fragment of sequence SEQ ID NO: 7 and (ii) the spin label MTSL.

Another purpose of the invention is to provide a diagnostic product, making possible the in vitro or in vivo diagnosis of diseases with abnormal expression or activity of ATP synthase or IF1. In fact, the overexpression of the protein IF1 can be observed in patients with certain cancers, in particular colon cancer, lung cancer, breast cancer or ovarian cancer, while abnormal activity of ATP synthase can be the cause of NARP syndrome (neuropathy, ataxia, retinitis pigmentosa) or Leigh's syndrome (MILS Maternally Inherited Leigh's Syndrome).

A diagnostic product of the invention contains at least one labelled polypeptide as described above.

The said diagnostic product of the invention can be utilised by means of various imaging techniques, such as scintigraphy (gamma radiation emission), the PET technique (positron emission tomography), fluorescence microscopy or MRI (magnetic resonance imaging).

The said diagnostic product can also contain a pharmaceutically acceptable excipient known to a person skilled in the art.

The said diagnostic product can be formulated in a form suitable for in vivo administration by oral route or by intravenous, intracranial, intratissue or intra-muscular injection.

The present invention also relates to a diagnostic kit making it possible for in vitro diagnosis of diseases connected with the abnormal expression of ATP synthase or of IF1.

The said kit contains a diagnostic product according to the present invention, a sample as a negative control and/or a sample as a positive control.

The measurement of the abnormal expression of IF1 can be utilised in an in vitro method for the diagnosis of cancer, in particular of colon cancer, lung cancer, breast cancer or ovarian cancer in a human or animal subject.

According to the invention, the said method comprises the following essential steps:
(i) contacting a sample for diagnosis obtained from the said subject with a diagnostic product of the present invention described above,
(ii) comparison of the level of expression of the intrinsic IF1 protein in the said sample with a normal threshold value,
(iii) determination of the presence of cancerous cells in the said sample obtained from the said subject when the level of expression of the intrinsic IF1 protein in the said sample is greater than that normal threshold value.

The labelled polypeptide of the invention enables the detection of the level of expression of the intrinsic IF1 protein by the formation of a complex with the intrinsic IF1 protein. The protein IF1 forms dimers or tetramers in vivo depending on the different pH conditions.

In said method, by “normal threshold value” is meant an average value of the level of expression of the intrinsic IF1 protein obtained in a pool of samples taken in persons not exhibiting any type of cancer.

The said sample for diagnosis can be a tissue or blood sample taken from a patient suspected to be developing a cancer.

The present invention also proposes a method for the in vitro diagnosis of a disease connected with abnormal activity of ATP synthase, in particular of NARP syndrome (neuropathy, ataxia, retinitis pigmentosa) or Leigh's syndrome (MILS Maternally Inherited Leigh's Syndrome) in a human subject.

According to the invention, the said method comprises the following essential steps:

(i) contact of a sample for diagnosis obtained from the said subject with a diagnostic product of the present invention described above,

(ii) comparison of the affinity of the labelled polypeptide of the invention in the said diagnostic product for the ATP synthase in the said sample with a normal threshold value,

(iii) determination of the abnormal activity of the ATP synthase in the said sample obtained from the said subject when the activity of the ATP synthase in the said sample is different from that normal threshold value.

In the said method, by “normal threshold value” is meant an average value of the affinity of the said labelled polypeptide for ATP synthase obtained in a pool of samples taken from persons exhibiting neither NARP syndrome, nor MILS syndrome.

The synthetic or hydrolytic activity of the ATP synthase in the said sample is determined by the affinity of a labelled polypeptide of the invention for this enzyme. An increase in the affinity between the ATP synthase and the said labelled polypeptide relative to the normal threshold value signifies a reduction in the activity of the ATP synthase relative to its normal activity. On the other hand, a decrease in the affinity between the ATP synthase and the said labelled polypeptide represents an increase in the activity of the ATP synthase.

The present invention also proposes a method for screening for a compound inhibiting ATPase, or a compound increasing the affinity of IF1 for The ATPase, comprising a step of measurement of the affinity between a labelled polypeptide of the invention and the said ATPase.

According to the invention, the said method comprises the following essential steps:

(i) contact of a labelled polypeptide according to the present invention with the ATPase in the presence of the compound to be tested,

(ii) comparison of the affinity between the said labelled polypeptide and the ATPase in the presence of the said compound with the affinity between the said labelled polypeptide and the ATPase in the absence of the said compound, in which when the affinity between the said labelled polypeptide and the ATPase in the presence of the said compound is less than that in the absence of the said compound, the said compound is identified as an inhibitor of the ATPase, and when the affinity between the said labelled polypeptide and the ATPase in the presence of the said compound is greater than that in the absence of the said compound, the said compound is identified as a compound increasing the affinity of IF1 for The ATPase.

The affinity between the said labelled polypeptide and the ATPase is measured by any technique known to a person skilled in the art, in particular according to the method described in the section “Materials and Methods” below.

The present invention also relates to a method for the in vitro or in vivo measurement of the level of expression of the ATPase in a subject, the said method comprising contacting a labelled polypeptide according to the present invention with a sample to be measured obtained from the said subject.

The level of expression of the ATPase is determined by its affinity with the said labelled polypeptide of the invention.

The present invention is now described in more detail and illustrated by Examples 1 to 3 below.

### EXAMPLE 1

### Production of a Labelled Polypeptide

1. Production of the Mutated Peptide Fragment of IF1

The nucleic acids encoding the mutated peptide fragments of IF1, namely SEQ ID NO: 7 (H27R-sc), SEQ ID NO: 9 (F15A-sc), SEQ ID NO: 10 (F15W-sc), were obtained by directed mutagenesis carried out using the Quickchange® kit (Stratagene) or the Phusion® polymerase.

The aforesaid nucleic acids are respectively inserted into the plasmid pET30a+ (Novagen) using the restriction enzyme in order to obtain the plasmids pET30a+H27R-sc, pET30a+F15A-sc, pET30a+F15W-sc.

1.1 Production of a Polypeptide Labelled with GFP

1.1.1 Cloning

The nucleic acid encoding the fluorescent protein GFP was cloned into the plasmid pET30a(+)-H27R-sc at the 5′ end of the nucleic acid encoding the peptide H27R-sc (SEQ ID NO: 7). For this cloning, two restriction sites BamH1 and NdeI were used. The BamH1 site was introduced by PCR at the 5′ end of the GFP gene and at the 5′ end of the NdeI site situated in pET30a+H27R-sc. The nucleic acid encoding the peptide spacer of sequence SEQ ID NO: 22 (L1) is added beforehand at the 3′ end of the nucleic acid encoding GFP by PCR. After transformation of XL1-Blue bacteria by thermal shock at 42° C. for 42 seconds, the positive clones containing the construct pET30a(+)GFP-L1-H27R-sc are selected for growth in a liquid selective LB medium (30 μg/mL of kanamycin), overnight at 37° C. The genome constructs are extracted, purified by miniprep (Qiagen GmbH/Macherey-Nagel) then checked by sequencing (Eurofins MWG Operon).

The plasmid pET30a(+)GFP-L1-H27R-sc obtained according to this protocol makes it possible to express the polypeptide of sequence SEQ ID NO: 24.

Another approach consists of firstly constructing by PCR a nucleic acid encoding a mutated peptide fragment of IF1 and a spacer. This nucleic acid is then cloned into the vector pET30a+ in order to obtain the vector pET30a+IF1-mutated-spacer. The nucleic acid encoding the GFP protein is then inserted into this vector.

When the nucleic acid encoding the GFP protein is cloned into the plasmid pET30(a)-H27R-sc at the 3′ end of the nucleic acid encoding the peptide H27R-sc (SEQ ID NO: 7), the peptide spacer of sequence SEQ ID NO: 21 (L2) is added beforehand by PCR at the 5′ end of the nucleic acid encoding GFP.

The plasmid pET30a(+)H27R-sc-L2-GFP obtained according to the protocol makes it possible to express the polypeptide of sequence SEQ ID NO: 23.

1.1.2 Transformation of Positive Clones by Electroporation

The plasmids containing the construct pET30a(+)GFP-L1-H27R-sc or pET30a(+)H27R-sc-L2-GFP are introduced by electroporation into Escherichia coli BL21(DE3), a competent bacterial heterologous expression system. The transformed bacteria grow overnight at 37° C. on a Petri dish containing 20 mL of LB+30 μg/mL of kanamycin.

1.1.3 Overexpression of Peptide

A colony is reinoculated into 10 mL of LB containing 30 μg/mL of kanamycin. After 12 hours of culturing at 37° C., 5 mL of culture are taken up in 500 mL of LB. The overexpression of the peptide GFP-L1-H27R-sc (SEQ ID NO: 24) or H27R-sc-L2-GFP (SEQ ID NO: 23) is induced by the addition of 0.5 mM IPTG once the OD_{600 nm} reaches a value lying between 0.6 and 0.8. After 3 hours of overexpression, the culture media are centrifuged for 20 mins at 4000 rpm. The pellets are then taken up in buffer A (0.3M NaCl, 50 mM Na₂HPO₄ adjusted to pH 6.5) containing 10 mM imidazole. The overexpression of peptide is monitored by 12% SDS-PAGE gel analysis containing protein samples “before” and “after overexpression” denatured at 95° C. for 3 minutes.

1.1.4 Purification of Peptide

The cells are firstly lysed by incubation for one hour with gentle stirring in a reagent solution containing 10 μg/mL DNase and RNase, 0.5 mg/mL lysozyme, and 1 mM PMSF (phenylmethylsulphonyl fluoride). Next, the cells are sonicated in ice for 3×45 seconds with 2 minute intervals between each sonication, then aliquotted into 4 mL volumes.

4 mL of cell lysate is then introduced into an Ni-NTA column previously equilibrated with buffer A containing 10 mM imidazole, then contacted with the column for 1 hour at ambient temperature. After 2 successive washings with 10 mM imidazole and a buffer A containing 20 mM imidazole, the peptide GFP-L1-H27R-sc (SEQ ID NO: 24) or the peptide H27R-sc-L2-GFP (SEQ ID NO: 23) are eluted by a buffer A containing 250 mM imidazole. The fractions containing the peptides of interest are combined then dialysed for 7 hours at 5° C., in dialysis buffer containing 50 mM NaCl, 20 mM Tris, pH 7.8. The proteins are then concentrated by Centricon Ultrafreer®-0.5 Centrifugal Filter Device (cutoff=5000) and stored at −20° C.

The cysteine-containing peptide of sequence SEQ ID NO: 25 has an enterokinase cleavage site between the sequence encoding the extension N-ter His₍₆₎ and that of the peptide of interest. After 2 successive washings with a tampon A containing 10 mM imidazole and a buffer A containing 20 mM imidazole, 30 U of enterokinase are added to the column in cleavage buffer containing 20 mM Tris and 50 mM NaCl adjusted to pH 7.4. After contact for 20 hours with the column at ambient temperature, the extension N-ter His₍₆₎ is dissociated from the protein of interest, and the latter is eluted starting with the first fractions. The fractions containing the protein of interest are combined, heated at 90° C. for 10 minutes, then centrifuged in a benchtop centrifuge for 20 minutes at 10° C. in order to remove the enterokinase. The proteins contained in the supernatant are then precipitated with TCA (10%). After centrifugation, the pellet is dissolved in 50 mM Na acetate, pH 5.5 and finally the dissolved peptides are stored at −20° C.

1.2 Production of a Polypeptide Labelled with MTSL

The nucleic acid encoding the peptide fragment of sequence SEQ ID NO: 25 was obtained by insertion of a cysteine codon into the median region of the nucleic acid encoding the peptide fragment of sequence SEQ ID NO: 9. The cysteine codon is introduced into the nucleic acid encoding the peptide fragment of sequence SEQ ID NO: 25 by PCR. The said cysteine is reduced so that a disulphide bridge can form between the peptide and the spin label MTSL. For the reduction of the cysteines, the whole of the buffers used is degassed under argon. 5 mM of TCEP (tris-(2-carboxyethyl)phosphine) are dissolved in 50 mM of MES (2-(N-morpholino)ethanesulphonic acid) buffer pH 6.5 and react with 40-80 μM of the peptide fragment of sequence SEQ ID NO: 25 for 40 minutes under a current of argon. The TCEP is then removed by passing the peptide through a PD10-G25 column. After harvesting the samples containing the protein (absorbance 280 nm), the label, the concentration whereof is 10 times greater than that of the peptide to be labelled, is added to the reaction medium and the reaction of labelling the protein with the spin label (MTSL) then takes place for 40 mins in an ice bath. Following the labelling, the excess MTSL is removed by passing the sample containing the protein through a PD10-G25 column.

### EXAMPLE 2

### Measurement of the Hydrolysis of ATP

The hydrolysis of ATP by F1-ATPase or SMP (F0F1-ATPase) was measured according to the reaction illustrated below by following the disappearance of NADH over time at 340 nm.

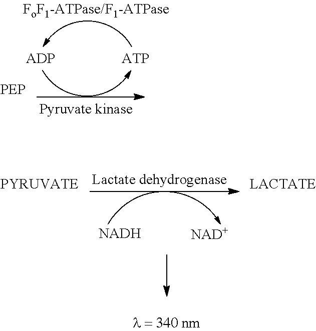

This reaction is carried out in a thermostatically controlled vessel (25° C.) in which the reaction medium pH 6.5 (50 mM MES, 20 mM KCl, 1 mM MgCl2, 0.4 mM NADH, 1 mM PEP, 20 units/mL pyruvate kinase and 50 unit/mL lactate dehydrogenase) is stirred. In the case of measurement of hydrolysis of ATP by the SMPs, the vessel also contains 3 μM antimycin and 3 μM FCCP.

### EXAMPLE 3

### Measurement of the Affinity of a Labelled Polypeptide for the ATPase and Processing of Data

After recording for 2 to 4 minutes, a labelled polypeptide of the invention is injected into the measurement vessel at a concentration range of 15 nM to 2 μM and the ATPase activity decreases. The recordings are analysed and adjusted with a decreasing monoexponential function (equation 1 shown below) corresponding to the decline in the ATPase activity.

$$y ( t ) = {V}_{∞} ( t ) + ( \frac{{V}_{0} - {V}_{∞}}{{k}_{app}} ) ( 1 - {e}^{- {k}_{app} t} ) + {y}_{0} \qquad Equation 1$$

In this equation: y(t) represents the absorbance at time t.

y₀ represents the absorbance at time 0, at the time of injection of the said labelled polypeptide.

V₍₀₎ represents the initial ATP hydrolysis rate before injection of the said labelled polypeptide.

V₍₁₎ represents the final rate after the addition of the said labelled polypeptide.

kₐₚₚ represents the apparent inhibition rate constant.

By plotting the values of kₐₚₚ obtained as a function of the concentration of IF1, the formation rate constant (kₒₙ) and dissociation rate constant (k_{off}) of the inhibited complex F1ATPase/IF1 are determined according to the following equation: kₐₚₚ=kₒₙ[I]+k_{off}

The relationship between the ratio V(I)/V(0) and the concentration of labelled polypeptide of the invention are linked according to the following equation:

V(I)/V(0)=Vᵣ+(1−Vᵣ)/(1+[I]/k₁),

in which Vᵣ is the fraction of V0 insensitive to IF1 inhibition.

The kinetic results obtained with the peptide H27R-sc-L2-GFP (SEQ ID NO: 23), GFP-L1-H27R-sc (SEQ ID NO: 24) or the cysteine-containing peptide of sequence (SEQ ID NO: 25) show that the labelled polypeptides of the invention exhibit a better affinity for ATP synthase compared to that of natural IF1.

1. Labelled polypeptide constituted by:
(i) a peptide fragment of 38 to 50 amino acids comprising a peptide of sequence SEQ ID NO: 1, and
(ii) a label selected from the group composed of radioactive groups, non-protein fluorophors, protein fluorophors, magnetic particles or a paramagnetic spin label, in order that when the said label is a fluorophor protein the said labelled polypeptide further comprises a peptide spacer of 3 to 15 amino acids, located between the said peptide fragment and the said label.

2. Labelled polypeptide according to claim 1, in which the said peptide fragment contains or is constituted by:
(i) a peptide mutated relative to a fragment of yeast IF1 of sequence SEQ ID NO: 2, the said mutated peptide containing at least one mutation at position 15, 27, 34 or 37 as defined relative to the 1^{st} amino acid of the sequence SEQ ID NO: 2, or
(ii) a peptide mutated relative to a fragment of bovine IF1 of sequence SEQ ID NO: 3, the said mutated peptide containing at least one mutation at position 19, 31, 42 or 45 as defined relative to the 1^{st} amino acid of the sequence SEQ ID NO: 3, or
(iii) a peptide mutated relative to a fragment of human IF1 of sequence SEQ ID NO: 3, the said mutated peptide containing at least one mutation at position 19, 31, 42 or 45 as defined relative to the 1^{st} amino acid of the sequence SEQ ID NO: 4.

3. Labelled polypeptide according to claim 1, in which the said peptide fragment contains or is constituted by a peptide represented by a sequence selected from: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a sequence having at least 80% similarity with an aforesaid sequence.

4. Labelled polypeptide according to claim 1, in which:
the said peptide spacer is an oligopeptide of 3 to 5 amino acids when the said spacer is located at the C-terminal end of the said peptide fragment,
the said peptide spacer is an oligopeptide of 9 to amino acids when the said spacer is located at the N-terminal end of the said peptide fragment.

5. Labelled polypeptide according to claim 4, selected from:
(a) a labelled polypeptide constituted, from the N-terminal end to the C-terminal end, by:
(i) a peptide fragment of 38 to 50 amino acids comprising a peptide of sequence SEQ ID NO: 1,
(ii) a peptide spacer of 3 to 5 amino acids, and
(iii) a fluorophor protein,
in particular a labelled polypeptide constituted, from the N-terminal end to the C-terminal end by:
(i′) a peptide fragment of sequence SEQ ID NO: 7, 9 or 10,
(ii′) a peptide spacer of sequence SEQ ID NO: 21,
(iii′) the protein GFP,
more particularly, a labelled polypeptide of sequence SEQ ID NO: 23, or
(b) a labelled polypeptide constituted, from the N-terminal end to the C-terminal end, by:
(i) a fluorophor protein,
(ii) a peptide spacer of 9 to 15 amino acids,
(iii) a peptide fragment of 38 to 50 amino acids comprising a peptide of sequence SEQ ID NO: 1,
in particular a labelled polypeptide constituted by, from the N-terminal end to the C-terminal end by:
(i′) the protein GFP,
(ii′) a peptide spacer of sequence SEQ ID NO: 22,
(iii′) a peptide fragment of sequence SEQ ID NO: 7, 9 or 10,
more particularly, a labelled polypeptide of sequence SEQ ID NO: 24.

6. Labelled polypeptide according to claim 1, constituted either by a peptide fragment of sequence SEQ ID NO: 7 radioactively labelled with a radioisotope selected from tritium, ³²P, ¹⁵O and ³⁵S, or by: (i) a peptide fragment of sequence SEQ ID NO: 7 and (ii) a chemical reagent bearing a radioisotope selected from tritium, ³²P, ¹⁵O and ³⁵S, or constituted by: (i) a peptide fragment of sequence SEQ ID NO: 7 and (ii) the fluorophor Dylight® 680, or
constituted by: (i) a peptide fragment of sequence SEQ ID NO: 25 and (ii) the spin label MTSL (S-(2,2,5,5-tetramethyl-2,5-dihydro-1H-pyrrol-3-yl)-methyl methanesulphonothioate).

7. Diagnostic product for a disease linked with abnormal activity of ATP synthase or of IF1, in particular a product intended for in vitro diagnosis of a cancer, such as colon cancer, lung cancer, breast cancer and ovarian cancer, or NARP (neuropathy, ataxia, retinitis pigmentosa) syndrome or MILS (Maternally Inherited Leigh's Syndrome) syndrome, characterised in that the said product contains at least one labelled polypeptide according to claim 1.

8. Method for in vitro diagnosis of a disease linked with abnormal activity of ATP synthase, in particular NARP syndrome or MILS syndrome, in a subject, comprising the following essential steps:
(i) contact of a sample for diagnosis obtained from the said subject with a diagnostic product according to claim 7,
(ii) comparison of the affinity of the labelled polypeptide of the invention in the said diagnostic product for ATP synthase in the said sample with a normal threshold value,
(iii) determination of the abnormal activity of the ATP synthase in the said sample obtained from the said subject when the activity of the ATP synthase in the said sample is different from that normal threshold value.

9. Method for screening for a compound inhibiting an ATPase or for a compound increasing the affinity of IF1 for The ATPase, characterised in that it comprises the following essential steps:
(i) contact of a labelled polypeptide according to claim 1 with the ATPase in the presence of the said compound,
(ii) comparison of the affinity between the said labelled polypeptide and the ATPase in the presence of the said compound with the affinity between the said labelled polypeptide and the ATPase in the absence of the said compound,
in which when the affinity between the said labelled polypeptide and the ATPase in the presence of the said compound is less than that in the absence of the said compound, the said compound is identified as an inhibitor of the ATPase; and when the affinity between the said labelled polypeptide and the ATPase in the presence of the said compound is greater than that in the absence of the said compound, the said compound is identified as a compound increasing the affinity of IF1 for the ATPase.

10. Method for in vitro measuring the level of expression of the ATPase in a subject, characterised in that it comprises the contact of a labelled polypeptide according to claim 1 with a sample to be measured obtained from the said subject.

11. Labelled polypeptide according to claim 2, in which the said peptide fragment contains or is constituted by a peptide represented by a sequence selected from: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a sequence having at least 80% similarity with an aforesaid sequence.

12. Labelled polypeptide according to claim 2, in which:
the said peptide spacer is an oligopeptide of 3 to 5 amino acids when the said spacer is located at the C-terminal end of the said peptide fragment,
the said peptide spacer is an oligopeptide of 9 to amino acids when the said spacer is located at the N-terminal end of the said peptide fragment.

13. Labelled polypeptide according to claim 3, in which:
the said peptide spacer is an oligopeptide of 3 to 5 amino acids when the said spacer is located at the C-terminal end of the said peptide fragment,
the said peptide spacer is an oligopeptide of 9 to amino acids when the said spacer is located at the N-terminal end of the said peptide fragment.

14. Labelled polypeptide according to claim 2, constituted either by a peptide fragment of sequence SEQ ID NO: 7 radioactively labelled with a radioisotope selected from tritium, ³²P, ¹⁵O and ³⁵S, or by: (i) a peptide fragment of sequence SEQ ID NO: 7 and (ii) a chemical reagent bearing a radioisotope selected from tritium, ³²P, ¹⁵O and ³⁵S, or constituted by: (i) a peptide fragment of sequence SEQ ID NO: 7 and (ii) the fluorophor Dylight® 680, or
constituted by: (i) a peptide fragment of sequence SEQ ID NO: 25 and (ii) the spin label MTSL (S-(2,2,5,5-tetramethyl-2,5-dihydro-1H-pyrrol-3-yl)-methyl methanesulphonothioate).

15. Labelled polypeptide according to claim 3, constituted either by a peptide fragment of sequence SEQ ID NO: 7 radioactively labelled with a radioisotope selected from tritium, ³²P, ¹⁵O and ³⁵S, or by: (i) a peptide fragment of sequence SEQ ID NO: 7 and (ii) a chemical reagent bearing a radioisotope selected from tritium, ³²P, ¹⁵O and ³⁵S, or constituted by: (i) a peptide fragment of sequence SEQ ID NO: 7 and (ii) the fluorophor Dylight® 680, or
constituted by: (i) a peptide fragment of sequence SEQ ID NO: 25 and (ii) the spin label MTSL (S-(2,2,5,5-tetramethyl-2,5-dihydro-1H-pyrrol-3-yl)-methyl methanesulphonothioate).


SEQUENCE LISTING (25 sequences)

SEQ ID NO: 1 - protein, 46 aa, artificial sequence
  consensus sequence
XXXXXXXXFXKREXAXEXXXXXXXXXEXLXXLXXXXXXXXXXXXXX

SEQ ID NO: 2 - protein, 38 aa, Saccharomyces cerevisiae
SEDSFVKRARATEDFFVRQREKEQLRHLKEQLEKQRKK

SEQ ID NO: 3 - protein, 46 aa, bos taurus
AVRDAGGAFGKREQAEEERYFRARAKEQLAALKKHKENEISHHAKE

SEQ ID NO: 4 - protein, 46 aa, homo sapiens
SIREAGGAFGKREQAEEERYFRAQSREQLAALKKHHEEEIVHHKKE

SEQ ID NO: 5 - protein, 38 aa, Saccharomyces cerevisiae
SEDSFVKRARATEDFFVRQREKEQLRHLKEQLEAQRKK

SEQ ID NO: 6 - protein, 38 aa, Saccharomyces cerevisiae
SEDSFVKRARATEDFFVRQREKEQLRHLKEQLEKQRAK

SEQ ID NO: 7 - protein, 38 aa, Saccharomyces cerevisiae
SEDSFVKRARATEDFFVRQREKEQLRRLKEQLEKQRKK

SEQ ID NO: 8 - protein, 38 aa, Saccharomyces cerevisiae
SEDSFVKRARATEDFFVRQREKEQLRALKEQLEKQRKK

SEQ ID NO: 9 - protein, 38 aa, Saccharomyces cerevisiae
SEDSFVKRARATEDWFVRQREKEQLRHLKEQLEKQRKK

SEQ ID NO: 10 - protein, 38 aa, Saccharomyces cerevisiae
SEDSFVKRARATEDAFVRQREKEQLRHLKEQLEKQRKK

SEQ ID NO: 11 - protein, 46 aa, bos taurus
AVRDAGGAFGKREQAEEERYFRARAKEQLAALKKHKENEISKHAKE

SEQ ID NO: 12 - protein, 46 aa, bos taurus
AVRDAGGAFGKREQAEEERYFRARAKEQLAALKKHKENEISHHAAE

SEQ ID NO: 13 - protein, 46 aa, bos taurus
AVRDAGGAFGKREQAEEERYFRARAKEQLARLKKHKENEISHHAKE

SEQ ID NO: 14 - protein, 46 aa, bos taurus
AVRDAGGAFGKREQAEEERYFRARAKEQLAHLKKHKENEISHHAKE

SEQ ID NO: 15 - protein, 46 aa, bos taurus
AVRDAGGAFGKREQAEEEFYFRARAKEQLAALKKHKENEISHHAKE

SEQ ID NO: 16 - protein, 46 aa, homo sapiens
SIREAGGAFGKREQAEEERYFRAQSREQLAALKKHHEEEIVKHKKE

SEQ ID NO: 17 - protein, 46 aa, homo sapiens
SIREAGGAFGKREQAEEERYFRAQSREQLAALKKHHEEEIVHHKAE

SEQ ID NO: 18 - protein, 46 aa, homo sapiens
SIREAGGAFGKREQAEEERYFRAQSREQLARLKKHHEEEIVHHKKE

SEQ ID NO: 19 - protein, 46 aa, homo sapiens
SIREAGGAFGKREQAEEERYFRAQSREQLAHLKKHHEEEIVHHKKE

SEQ ID NO: 20 - protein, 46 aa, homo sapiens
SIREAGGAFGKREQAEEEFYFRAQSREQLAALKKHHEEEIVHHKKE

SEQ ID NO: 21 - protein, 4 aa, artificial sequence
  L2 espacer
GGSG

SEQ ID NO: 22 - protein, 15 aa, artificial sequence
  L1 espacer
GGGGSGGGGSGGGGS

SEQ ID NO: 23 - protein, 280 aa, artificial sequence
  GFP marked polypeptide
SEDSFVKRARATEDFFVRQREKEQLRRLKEQLEKQRKKGGSGMSKGEELFTGVVPILVELDGDVNGHKFSVSGEGEGDATYGKLTLKFICTTGKLPVPWPTLVTTFAYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSVLSKDPNEKRDHMVLLEFVTAAGITHGMDELYK

SEQ ID NO: 24 - protein, 291 aa, artificial sequence
  GFP marked polypeptide
MSKGEELFTGVVPILVELDGDVNGHKFSVSGEGEGDATYGKLTLKFICTTGKLPVPWPTLVTTFAYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSVLSKDPNEKRDHMVLLEFVTAAGITHGMDELYKGGGGSGGGGSGGGGSSEDSFVKRARATEDFFVRQREKEQLRRLKEQLEKQRKK

SEQ ID NO: 25 - protein, 39 aa, artificial sequence
  cysteine polypeptide
SEDSFVKRARATEDWFVRQREKECQLRHLKEQLEKQRKK